(19) United States
(12) Patent Application Publication
(10) Pub. No.: US 2007/0162312 A1
(43) Pub. Date: Jul. 12, 2007

(54) Physician Treatment Ordering System

(75) Inventor: James Barton Soderberg, Sandy, UT (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS MEDICAL SOLUTIONS HEALTH SERVICES CORPORATION, MALVERN, PA (US)

(21) Appl. No.: 11/538,515

(22) Filed: Oct. 4, 2006

Related U.S. Application Data
(60) Provisional application No. 60/737,343, filed on Nov. 16, 2005.

(51) Int. Cl. G06F 19/00; A61B 5/00

(52) U.S. Cl. 705/3; 600/300

(57) ABSTRACT

A system provides decision support information that improves patient care by enabling physicians who are placing orders to see historical treatment ordering patterns and adjust order associated workflow (e.g., as part of a computerized Health Care Order Entry System) based on an analysis of past orders placed on similar types of patients with similar demographic information under similar conditions. A patient treatment decision support system employing historical treatment order data includes a pre-processor. The pre-processor analyzes and collates historical patient treatment order information for multiple different patients based on criteria including diagnostic code to provide historical order information identifying treatment orders previously initiated for patients associated with a treatment order of a particular type. The historical treatment order information is stored by the pre-processor in a repository. A user interface receives user entered data identifying a particular treatment order for a particular patient having particular characteristics and provides a display of data identifying historical order information associated with the particular treatment order by deriving the historical order information from the repository based on a determined type of the particular treatment order.

### CROSS-REFERENCE TO RELATED APPLICATIONS

The present application is a non-provisional application of provisional application having Ser. No. 60/737,343 by J. B. Soderberg on Nov. 16, 2005.

### FIELD OF THE INVENTION

The present invention concerns a patient treatment decision support system employing order type information and diagnostic codes in deriving and processing historical order information.

### BACKGROUND OF THE INVENTION

In existing healthcare enterprises, there are occasions when patient care is compromised because an order for a treatment for a patient is not placed and a laboratory test for the patient is not performed. This occurs because of ignorance, or because of human error. Some existing systems use treatment Protocols determining best practice treatment processes for a particular patient medical condition. However, treatment Protocols are not commonly used because of the difficulty involved in their creation. A Protocol needs to be agreed upon by a group of users. It is usually difficult for the framers of a protocol to come to a consensus of what should be included and it takes a long time to reach consensus. Because of this, few protocols get put into practice. A system according to invention principles addresses these deficiencies and related problems.

### SUMMARY OF THE INVENTION

system provides decision support information that improves patient care by enabling physicians initiating treatment orders to see an analysis of orders and order patterns that have historically been placed for similar types of patients with similar demographic information under similar conditions. A patient treatment decision support system employing historical treatment order data includes a pre-processor. The pre-processor analyzes and collates historical patient treatment order information for multiple different patients based on criteria including diagnostic code to provide historical order information identifying treatment orders previously initiated for patients associated with a treatment order of a particular type. The historical treatment order information is stored by the preprocessor in a repository. A user interface receives user entered data identifying a particular treatment order for a particular patient having particular characteristics and provides a display of data identifying historical order information associated with the particular treatment order by deriving the historical order information from the repository based on a determined type of the particular treatment order.

### BRIEF DESCRIPTION OF THE DRAWINGS

FIG. 1 shows a system for processing historical patient treatment order data to provide collated, indexed historical treatment order data, in accordance with invention principles.

FIG. 2 shows a system for processing a patient treatment order using collated, indexed historical treatment order data, in accordance with invention principles.

FIG. 3 shows a system for validating a patient treatment order, in accordance with invention principles.

FIG. 4 shows an order entry display image menu providing a user access to collated, indexed historical treatment order data based on predetermined criteria, in accordance with invention principles.

FIG. 5 shows an order entry display image menu illustrating accessed collated, indexed historical treatment order data, in accordance with invention principles.

FIG. 6 shows a patient treatment decision support system employing historical treatment order data, in accordance with invention principles.

### DETAILED DESCRIPTION OF THE INVENTION

A system according to invention principles provides insight into common historical treatment ordering practices and allows physicians to see laboratory tests and orders that have been placed in the past. The system analyzes current and past order communication transactions records and organizes the analyzed data by indexing the data within one or more repositories (databases). The current and past order communication transactions records are indexed by type of orders placed on patients with selected demographic characteristics (race, gender, age, weight, height, etc.,) admitting diagnosis, and co-morbidity codes. Other demographic characteristics include, Female Postmenopausal Status, Female Premenopausal Status, Pregnancy Indicator, Vaccination completed (such as Diphtheria, Tetanus, H. influenzae and Measles), Menstrual Cycle (including Early follicular phase, Late follicular phase and Luteal phase), Gestational Age, Pre-pubertal Status and Diabetic or Nondiabetic Status. In an example, the system analyzes laboratory tests ordered for a 35-40 year old female, with an admitting diagnosis of stomach cramps and co-morbidity of diabetes by grouping the laboratory tests. In addition, the system also determines the likelihood of these tests being ordered for this combination of diagnostic indicators and the particular patient demographic characteristics. Further, when an older is placed on a patient matching the particular patient demographic characteristics and diagnostic codes, the information (types of orders and likelihood of this order being placed on a patient of this type) is displayed to support clinical decision making. This data is optionally categorized by ordering user, ordering user group, specialty and department, etc.

The system provides organized results of an analysis of order communication transactions records to a user upon user initiation of a treatment order for a pre-selected patient entered via an Order Entry displayed image menu. The organized analysis results are generated in response to patient demographics, admission codes, and general health codes. The results are displayed in a separate image window in an order entry composite display image, for example, upon a user selecting a button in the order entry composite display image. In one embodiment, the organized analysis results are displayed in response to user selection of one of the options in an order-type drop-down box. In a further embodiment, the system uses the organized analysis results to pre-populate a selectable treatment Orders image window enabling a user to select an order for a patient from the organized analysis results.

The system also notifies a user, (before or after an order is placed) that certain historical tests (with high confidence levels) have not been ordered and should he considered for being ordered. The notification requires a physician to either add the order at this point or to click cancel. The system provides clinical decision support information that is independent of treatment protocols and is adaptable to select and use many different types of historical treatment data. The decision support information is based on historical treatment data and is not predefined or based on group consensus and the decision support information database grows and improves with use. The system advantageously reduces treatment ordering oversight and errors by making users aware of past practices. The organized analysis results generated by the system are also used to review user treatment ordering practices by patient types and the system may be configured to require a user to electronically indicate that they do not want to initiate highly significant orders. The system does not practice medicine or prescribe use of fixed ordering practices and is readily extended to other (e.g., non-healthcare) applications. The system employs many different sources of data for its analysis including current data, archived data and multiple different distributed databases accessed locally or remotely and is configurable to notify users, or others, when certain tests have not been ordered as well as to monitor ordering trends.

An executable application as used herein comprises code or machine readable instruction for implementing predetermined functions including those of an operating system, healthcare information system or other information processing system, for example, in response user command or input. An executable procedure is a segment of code (machine readable instruction), sub-routine, or other distinct section of code or portion of an executable application for performing one or more particular processes and may include performing operations on received input parameters (or in response to received input parameters) and provide resulting output parameters. A processor as used herein is a device and/or set of machine-readable instructions for performing tasks. As used herein, a processor comprises any one or combination of. hardware, firmware, and/or software. A processor acts upon information by manipulating, analyzing, modifying, converting or transmitting information for use by an executable procedure or an information device, and/or by routing the information to an output device. A processor may use or comprise the capabilities of a controller or microprocessor, for example. A display processor or generator is a known element comprising electronic circuitry or software or a combination of both for generating display images or portions thereof. A user interface comprises one or more display images enabling user interaction with a processor or other device. Further, a workflow is a sequence of tasks performed by human or device or a combination of both.

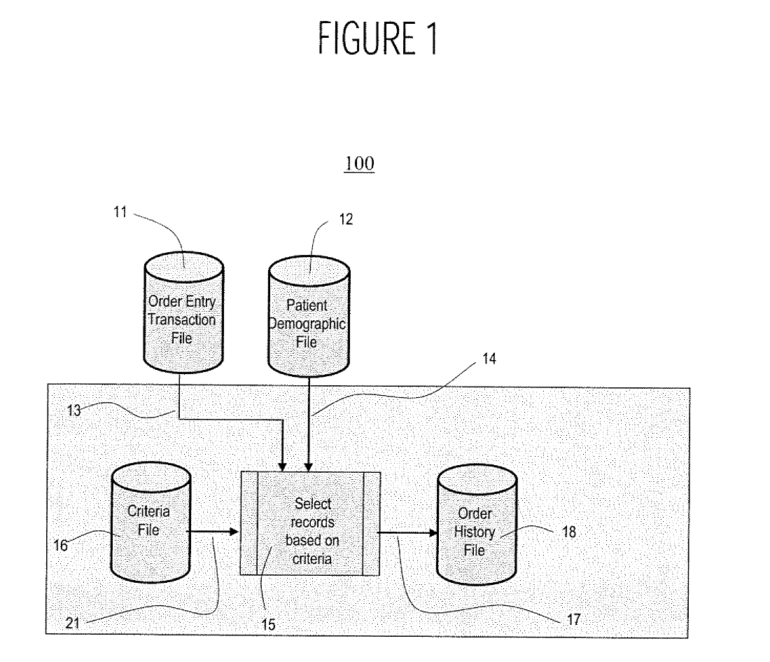

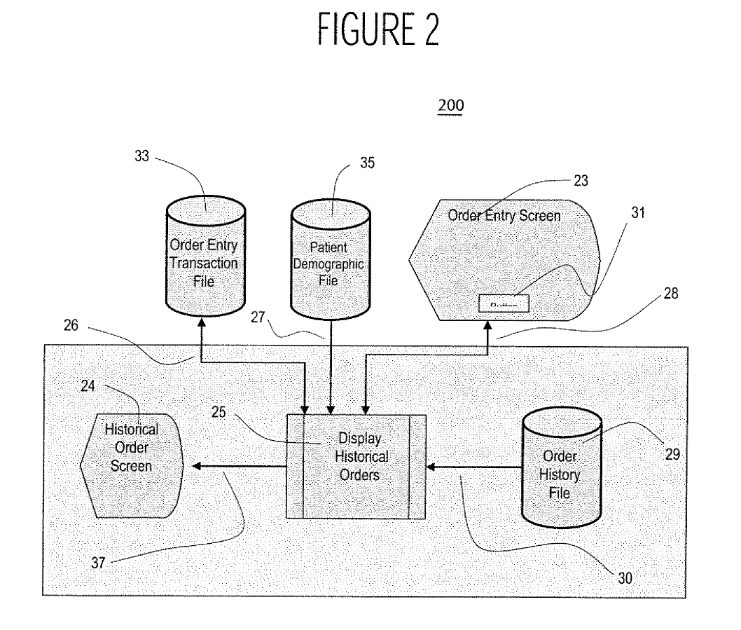

FIG. 1 shows system 100 for processing historical patient treatment order data to provide collated, indexed historical treatment order data. System 100 provides files of historical patient treatment order data for subsequent use by system 200 of FIG. 2 in processing a patient treatment order. Data files 11 and 12 are existing files including data comprising a treatment order record of a particular patient and demographic characteristics of the particular patient, respectively. Data files 11 and 12 are accessed via paths 13 and 14 respectively by Historical Analysis processor 15 and processed to generate a treatment Order History file. For this purpose, Historical Analysis processor 15 parses and examines Criteria file 16, accessed via path 21, to determine historical treatment order selection criteria of a hospital (or other organization or entity). As an example, a hospital organizes its historical treatment order data by Admission Code and co-morbidity values. Another different hospital may chose to further filter and restrict selected historical treatment order data by considering parameters such as patient age, sex, race, etc. Historical Analysis processor 15 acquires data comprising historical treatment orders that match the different criteria and variables indicated in criteria file 16 accessed via interface 21. In another embodiment, criteria file 16 may contain default filter and selection criteria. Specifically, Historical Analysis processor 15 parses acquired historical treatment order data to filter the data by identifying treatment orders that match the different criteria in file 16 and stores the acquired, filtered historical treatment order data via interface 17 in Order History file 18.

Historical Analysis processor 15 also parses and analyzes the acquired, filtered historical treatment order data by performing a statistical, probabilistic, percentage or other analysis indicated in criteria file 16 and stores analysis results in file 18. Historical Analysis processor 15, for example, identifies and stores treatment order records including at least one treatment order of a user selected group of orders and also calculates the percentage of patients of the total patient population in the database of historical order records for which at least one treatment order of the user selected group was placed. Historical Analysis processor 15 also calculates a standard statistical analysis value indicating a likelihood of at least one treatment order of the user selected group of orders needing to be placed on a particular patient. The analysis information generated by processor 15 is stored in the Order History File 18. System 100 advantageously performs an analysis to derive analysis information prior to a user initiating a related treatment order to increase system response time and eliminate the delay involved in performing the analysis after a user places a related treatment order.

FIG. 2 shows system 200 for processing a patient treatment order using collated, indexed historical treatment order data generated by system 100. In response to a physician command, Order Entry image 23 incorporating historical treatment order button 31 is displayed via a user interface. Upon user selection of button 31, Historical Orders function 25 initiates display of Historical order information image 24 via communication paths 28 and 37. Historical order information image 24 is generated by function 25. For this purpose, function 25 acquires data representing historical treatment order records from Order History file 29 via interface 30 that match criteria of the particular patient concerned. The particular patient criteria comprise a treatment order transaction record 33 of the particular patient and demographic characteristics file 35 of the particular patient, respectively. Function 25 acquires data representing historical treatment order records from History file 29 that match criteria in transaction record 33 and demographic characteristics file 35 of the particular patient accessed via communication paths 26 and 27, respectively. In providing Historical Order image 24, function 25 excludes those historical treatment orders in treatment order transaction record 33 of the particular patient that have been previously placed and administered to the particular patient and that are no longer currently active. Thereby, Historical order information image 24 indicates physician treatment orders that are currently active for administration to the particular patient and historical orders that have not been initiated for this patient. Historical order information image 24, also displays statistical values indicating a likelihood that a candidate treatment order selected for potential initiation for the particular patient using the order entry system should be confirmed and placed. Historical order information image 24 also presents a checkbox allowing a physician to confirm and order the test, treatment, object, etc. and other historical orders that have not yet been selected and initiated for the particular patient.

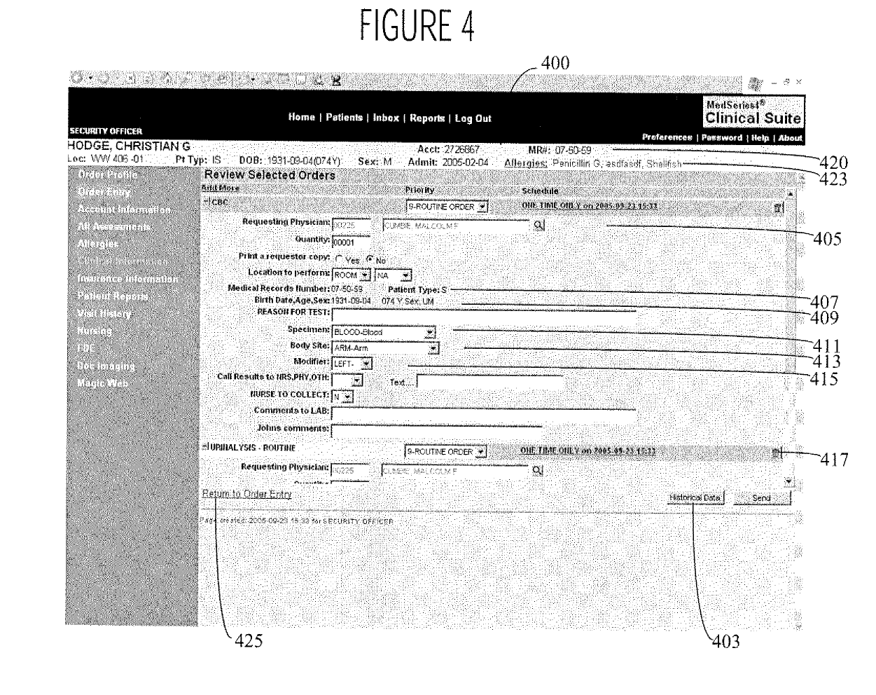

FIG. 4 shows order entry display image menu 400 enabling a requesting physician (identified in row 405) to review an order to be placed for a blood test (identified in rows 411, 413 and 415) for a patient (of patient type S identified in rows 407, 409, 420 and 423). Image menu 400 is accessed by a physician during a treatment order entry process as part of a physician order entry workflow and the physician returns to the order entry workflow upon selection of button 425. In response to user selection of button 403, system 200 (FIG. 2) accesses collated, indexed historical treatment order data based on predetermined criteria, as provided by system 100 (FIG. 1). In response to a physician clicking Historical Data button 403, a historical treatment order menu indicating historical orders that have been placed on this type of patient (e.g., patient type S having similar Admitting Diagnosis, co-morbidity, and other pre-defined criteria). System 100, for example, identifies and stores treatment order records for this type of patient and also calculates the percentage of patients of a total patient population in a database of historical order records for which the type of blood test (identified in rows 411, 413 and 415) was placed. System 100 also calculates a standard statistical analysis value indicating a likelihood of this blood test needing to be placed on the particular patient concerned based on treatment order transaction record 33 (FIG. 2) and demographic characteristics file 35 of the particular patient.

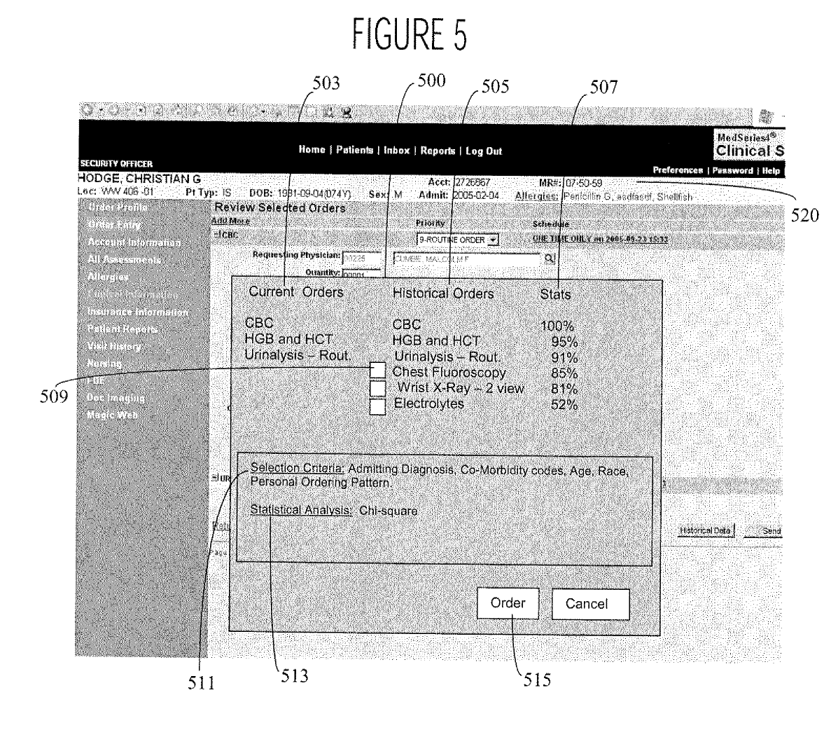

FIG. 5 shows an order entry display image menu 500 illustrating accessed collated, indexed historical treatment order data. Specifically, FIG. 5 shows a type of dialog box that is displayed if Historical Data button 403 (FIG. 4) is selected. Current candidate treatment orders to be placed using an order entry system are displayed in column 503. Treatment orders that have historically been placed on this type of patient are displayed in column 505. A check box prompt (e.g., 509) is displayed adjacent to those treatment orders that have not yet been ordered for the particular patient identified in row 520. The treatment orders that have not yet been ordered are added to a list of current candidate treatment orders identified in column 503 in response to user selection of associated check boxes (e.g., 509) and user selection of Order button 515. Thereby, a checked treatment order is added to the current candidate treatment orders (CBC, HGB and HCT and Urinalysis) identified in column 503 for initiation and processing for administration to the particular patient. A user configures Historical Analysis processor 15 of system 100 (FIG. 1) and/or Historical Orders function 25 of system 200 (FIG. 2) with historical order selection criteria. So, for example, Historical Orders function 25 filters out and excludes treatment orders that have been placed for the particular patient in the past and that are also found in the historical order list for the associated patient type. The treatment order selection criteria employed in providing the historical treatment order list of column 505 (FIG. 5) is indicated in item 511. Information concerning the statistical analysis used is indicated in item 513.

Column 507 provides statistics corresponding to associated historical treatment orders in column 505. Systems 100 and 200 may provide a wide variety and number of different types of statistics. Column 507 may indicate, for example, the percentage of patients of the total number of patients in the historical treatment order database that received the corresponding treatment order indicated in column 505. Column 507 may in addition provide a standard statistical analysis value indicating a likelihood of a corresponding treatment order identified in column 505 needing to be placed on the particular patient identified in row 520 (and patient type) concerned. A user configures systems 100 and 200 so that treatment orders that have statistical values lower than a predetermined user configured threshold level are not displayed in column 505 or, alternatively are displayed in column 505 but with no corresponding statistical value in column 507. These examples are illustrative only. One of ordinary skill in the art understands that there are many ways that the Historical data may be presented or made available to the physician.

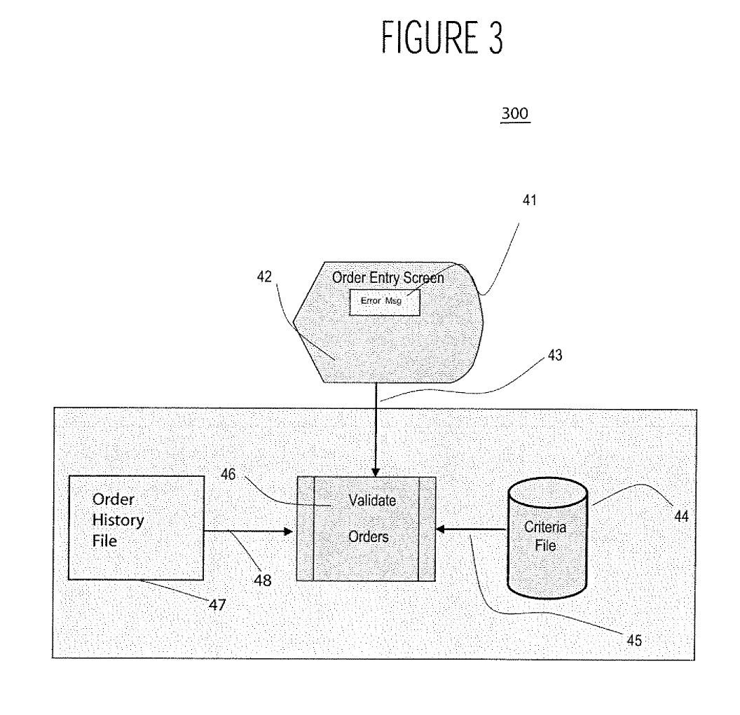

FIG. 3 shows system 300 for validating a patient treatment order and notifying a physician of historical treatment order information concerning a candidate treatment order. Within an order entry workflow during which a physician selects a candidate treatment order for a particular patient, error (alert) message 41 is displayed in order entry image menu 42 by order validation unit 46 via interface 43, in response to detection by unit 46 that the physician has failed to select one or more highly-significant historical orders related to the candidate order. Error message 41 informs the physician of the highly-significant historical treatment orders derived from order history file 47 via interface 48. These treatment orders are derived based on a treatment order transaction record of the particular patient, demographic characteristics of the particular patient as well as user-defined values in Criteria file 44 accessed via interface 45, in a similar manner to that described in connection with FIGS. 1 and 2. In another embodiment the highly-significant historical treatment orders are derived in real-time using an Order Entry Transaction file (not shown for drawing clarity) indicating current actual or proposed treatment orders. The physician responds to message 41 by selecting an acknowledgement response.

In another embodiment a historical treatment order analysis is performed in response to a physician accessing an Order entry image window or in response to other order entry functions prior to confirming selection of an order or during validation of a selected order. The system may also perform a different type of analysis involving more and different selection criteria as well as other significant patient criteria and a particular frequency of use of individual treatment orders. The system is usable for any other ordering process with other users, such as nurses, practitioners, administrative personnel, etc. Further, the analyzed data is usable in other ways. For example, it may be used to track physician ordering practices.

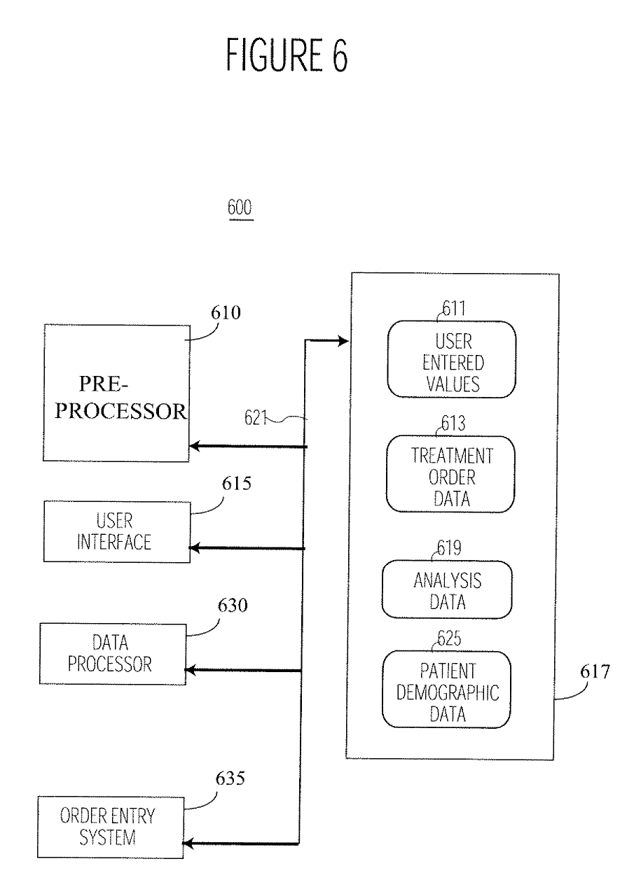

FIG. 6 shows patient treatment decision support system 600 employing historical treatment order data. System 600 comprises pre-processor 610, at least one repository 617, data processor 630 and order entry system 635 hi-directionally inter-communicating using communication path 621. The communication path 621 (otherwise called network, bus, link, connection, channel, etc.) may use any type of protocol or data format. The protocol or data format includes, but is not limited to, one or more of the following: an Internet Protocol (IP), a Transmission Control Protocol Internet protocol (TCPIP), a Hyper Text Transmission Protocol (HTTP), an RS232 protocol, an Ethernet protocol, a Medical Interface Bus (MIB) compatible protocol, a Local Area Network (LAN) protocol, a Wide Area Network (WAN) protocol, a Campus Area Network (CAN) protocol, a Metropolitan Area Network (MAN) protocol, a Home Area Network (HAN) protocol, an Institute Of Electrical And Electronic Engineers (IEEE) bus compatible protocol, a Digital and Imaging Communications (DICOM) protocol, and a Health Level Seven (HL7) protocol.

Pre-processor 610 analyzes and collates historical patient treatment order information for multiple different patients based on criteria including diagnostic code to provide historical treatment order information identifying treatment orders previously initiated for patients associated with a treatment order of a particular type. Pre-processor 610 stores the historical treatment order information in at least one repository 617 using network 621. Pre-processor 610 acquires the historical patient treatment order information for analysis and collation from treatment order transaction messages and one or more order information repositories (not shown). The treatment order transaction messages are generated by order entry system 635 enabling a user to select a candidate treatment order for a particular patient. Pre-processor 610 analyzes and collates historical patient treatment order information for multiple different patients based on criteria comprising particular characteristics including two or more of (a) age, (b) gender, (c) pre-existing medical condition, (d) weight, (e) height and (f) race.

Pre-processor 610 analyzes and collates the historical patient treatment transaction order information (and other treatment order information) by indexing identified treatment orders previously initiated for patients and associating indexed identified treatment orders with a treatment order of a particular type and/or a patient of a particular type. Pre-processor 610 pre-caches the historical treatment order information in at least one repository 617 to reduce access time to the historical patient treatment order information upon the user subsequently placing a related order, for example, by entering data identifying a particular treatment order for the particular patient using order entry system 635. Further, in response to predetermined limit data, e.g., comprising a treatment order storage size limit or number of order records limit, pre-processor 610 analyzes and collates a portion of the historical patient treatment order information to reduce processing time. In addition, pre-processor 610 adaptively provides the historical treatment order information by intermittently acquiring new historical patient treatment order information for analysis and collation.

Data processor 630 receives user entered data identifying a particular treatment order for a particular patient having particular characteristics from order entry system 635 and provides a display of data via user interface 615 identifying historical treatment order information associated with the particular treatment order. Data processor 630 does this by identifying and deriving, from at least one repository 617, the historical treatment order information comprising treatment orders 613 including, a diagnostic code common to both the particular treatment order and historical treatment orders and having demographic characteristics 625 substantially similar to both the particular patient and patients associated with historical treatment orders and excluding treatment orders that have been placed for the particular patient in the past and that are also found in historical treatment orders. Data processor 630 further identifies and derives, from at least one repository 617, the historical treatment order information 613 associated with the particular treatment order based on a user defined value 611 and by identifying a hospital admitting diagnosis common to both the particular patient and patients associated with historical treatment orders.

The display of data provided by data processor 630 via user interface 615 further includes statistical data (analysis data 619) concerning a corresponding historical treatment order. The statistical data 619 indicates a likelihood the corresponding historical treatment order needs to be placed and a proportion of patients of the total number of patients in a historical treatment order database that received the corresponding historical treatment order. The display of data provided by data processor 630 also includes a message informing a user of significant historical treatment orders, in response to detection by processor 630 that the user has failed to select one or more significant historical treatment orders related to a candidate treatment order selected using order entry system 635. User interface 615 also provides a display of data identifying historical treatment order information comprising individual treatment order types associated with a particular treatment order together with information identifying a proportion of a sampled patient population receiving the individual treatment order types. User interface 615 additionally provides a user selectable button in a display image supporting user entry of data provided by system 635 identifying a particular treatment order for a particular patient. The button enables initiation of display of a display image presenting data identifying historical treatment order information associated with a particular treatment order. The display image identifies criteria and a statistical function used in analyzing and collating the historical patient treatment order information. User interface 615 initiates generation of an alert message to a user indicating another treatment order has been placed together with said particular treatment order in a proportion of cases exceeding a predetermined threshold. The historical treatment order information associated with a particular treatment order is derived from at least one repository 617. This is done based on (a) determined type of particular treatment order indicating a particular patient has similar demographic characteristics to patients associated with historical treatment order information, (b) treatments identified in the historical treatment order information and a particular treatment order are associated with the same diagnostic code, (c) treatments identified in historical treatment order information and a particular treatment order are associated with the same hospital admitting diagnosis and (d) treatments identified in historical treatment order information and a particular treatment order are associated with the same co-morbidity.

The system and processes presented in FIGS. 1-6 are not exclusive, Other systems, processes and menus may be derived in accordance with the principles of the invention to accomplish the same objectives. Although this invention has been described with reference to particular embodiments, it is to be understood that the embodiments and variations shown and described herein are for illustration purposes only. Modifications to the current design may be implemented by those skilled in the art, without departing from the scope of the invention. A system according to invention principles is applicable anywhere that patient treatment decision support information is useful. Further, any of the functions provided in the systems of FIGS. 1-3 and 6 may be implemented in hardware, software or a combination of both and may reside on one or more processing devices located at any location of a network linking the FIG. 6 elements or another linked network including another intra-net or the Internet.

What is claimed is:

1. A patient treatment decision support system. employing historical treatment order data, comprising:
a pre-processor for analyzing and collating historical patient treatment order information for a plurality of different patients based on criteria including diagnostic code to provide historical treatment order information identifying treatment orders previously initiated for patients associated with a treatment order of a particular type, said historical treatment order information being stored by said pre-processor in a repository; and
a user interface for receiving user entered data identifying a particular treatment order for a particular patient having particular characteristics and for providing a display of data identifying historical treatment order information associated with said particular treatment order by deriving said historical treatment order information from said repository based on a determined type of said particular treatment order.

2. A system according to claim 1, wherein
said pre-processor analyzes and collates historical patient treatment order information for a plurality of different patients based on criteria comprising particular characteristics including two or more of, (a) age, (b) gender, (c) pre-existing medical condition, (d) weight, (e) height and (f) race.

3. A system according to claim 1, wherein
said historical patient treatment order information comprises transaction order information derived from a computerized treatment order entry system and
said pre-processor analyzes and collates said historical patient treatment transaction order information by indexing identified treatment orders previously initiated for patients and associating indexed identified treatment orders with a treatment order of a particular type.

4. A system according to claim 1, wherein
said user interface provides a display of data identifying historical treatment order information comprising individual treatment order types associated with said particular treatment order together with information identifying a proportion of a sampled patient population receiving said individual treatment order types.

5. A system according to claim 1, wherein
said preprocessor analyzes and collates said historical patient treatment order information to provide said historical treatment order information for pre-caching in said repository to reduce access time to said historical patient treatment order information upon said user subsequently entering said data identifying said particular treatment order for said particular patient.

6. A system according to claim 5, wherein
said pre-processor analyzes and collates a portion of said historical patient treatment order information in response to predetermined limit data to reduce processing time.

7. A system according to claim 1, wherein
said user interface provides a user selectable button in a display image supporting said user entry of said data identifying said particular treatment order for said particular patient, said button enabling initiation of display of a display image presenting said data identifying historical treatment order information associated with said particular treatment order.

8. A system according to claim 7, wherein
said display image identifies at least one of, (a) said criteria used in analyzing and collating said historical patient treatment order information to provide said historical treatment order information and (b) a statistical function used in analyzing and collating said historical patient treatment order information to provide said historical treatment order information.

9. A system according to claim 1, wherein
said user interface initiates generation of an alert message to a user indicating another treatment order has been placed together with said particular treatment order in a proportion of cases exceeding a predetermined threshold.

10. A system according to claim 1, wherein
said historical treatment order information associated with said particular treatment order is derived from said repository based on determined type of said particular treatment order indicating at least one of, (a) said particular patient has similar demographic characteristics to patients associated with said historical treatment order information, (b) treatments identified in said historical treatment order information and said particular treatment order are associated with the same diagnostic code, (c) treatments identified in said historical treatment order information and said particular treatment order are associated with the same hospital admitting diagnosis and (d) treatments identified in said historical treatment order information and said particular treatment order are associated with the same co-morbidity.

11. A patient treatment decision support system employing historical treatment order data, comprising:
a pre-processor for analyzing and collating historical patient treatment order information for a plurality of different patients based on criteria including diagnostic code to provide historical treatment order information identifying treatment orders previously initiated for patients associated with a treatment order of a particular type, said historical treatment order information being stored by said pre-processor in a repository; and
a data processor for receiving user entered data identifying a particular treatment order for a particular patient having particular characteristics and for providing a display of data identifying historical treatment order information associated with said particular treatment order by deriving said historical treatment order information from said repository, said historical treatment order information being derived by,
(a) identifying a diagnostic code common to both said particular treatment order and historical treatment orders and
(b) identifying demographic characteristics substantially similar to both said particular patient and patients associated with historical treatment orders.

12. A system according to claim 11, wherein
said historical treatment order information is derived based on a user defined value.

13. A system according to claim 11, wherein
said historical treatment order information is derived by identifying a hospital admitting diagnosis common to both said particular patient and patients associated with historical treatment orders.

14. A system according to claim 11, wherein
said display of data includes statistical data concerning a corresponding historical treatment order.

15. A system according to claim 14, wherein
said statistical data indicates at least one of, (a) a likelihood said corresponding historical treatment order needs to be placed and (b) a proportion of patients of the total number of patients in a historical treatment order database that received said corresponding historical treatment order.

16. A system according to claim 11, wherein
said data processor derives said historical treatment order information by excluding treatment orders that have been placed for the particular patient in the past and are also found in historical treatment orders.

17. A system according to claim 11, including
an order entry system enabling a user to select a candidate treatment order for a particular patient wherein
said data processor provides a display of data including a message informing a user of significant historical treatment orders, in response to detection that said user has failed to select one or more significant historical treatment orders related to said candidate treatment order.

18. A system according to claim 11, wherein
said pre-processor adaptively provides said historical treatment order information by intermittently acquiring new historical patient treatment order information for analysis and collation.

19. A system according to claim 11, including
an order entry system enabling a user to select a candidate treatment order for a particular patient wherein
said pre-processor acquires said historical patient treatment order information for analysis and collation from treatment order transaction messages generated by said order entry system.

20. A patient treatment decision support system employing historical treatment order data, comprising:
a pre-processor for analyzing and collating historical patient treatment order information for a plurality of different patients based on criteria including diagnostic code to provide historical treatment order information identifying treatment orders previously initiated for patients associated with a treatment order of a particular type, said historical treatment order information being stored by said pre-processor in a repository; and
a data processor for receiving user entered data identifying a particular treatment order for a particular patient having particular characteristics and for providing a display of data identifying historical treatment order information associated with said particular treatment order by deriving said historical treatment order information from said repository, said historical treatment order information comprising treatment orders including,
a diagnostic code common to both said particular treatment order and historical treatment orders and having demographic characteristics substantially similar to both said particular patient and patients associated with historical treatment orders and excluding treatment orders that have been placed for the particular patient in the past and are also found in historical treatment orders.

21. A system according to claim 20, wherein
said display of data includes statistical data concerning a corresponding historical treatment order and indicates a likelihood said corresponding historical treatment order needs to be placed.

22. A system according to claim 20, wherein
said display of data includes statistical data concerning a corresponding historical treatment order and indicates a proportion of patients of the total number of patients in a historical treatment order database that received said corresponding historical treatment order.
